(19) United States
(12) Patent Application Publication
(10) Pub. No.: US 2002/0039559 A1
(43) Pub. Date: Apr. 4, 2002

(54) Oral care composition

(76) Inventor: Paul H. Paek, Los Angeles, CA (US)

Correspondence Address: JOHN K. PARK, PARK & SUTTON LLP, SUITE 1110, 3255 WILSHIRE BLVD., LOS ANGELES, CA, 90010, US

(21) Appl. No.: 09/776,500

(22) Filed: Feb. 5, 2001

Related U.S. Application Data
(63) Continuation of application No. 09/637,162, filed on Aug. 14, 2000, now abandoned.

(51) Int. Cl. A61K 9/68; A61K 7/26; A61K 35/78

(52) U.S. Cl. 424/48; 424/58; 424/725

(57) ABSTRACT

An oral care composition comprising a Cimicifuga-salt, a coptis, a zanthoxylin, and a filler material is disclosed. The Cimicifuga-salt consists of a salt and a Cimicifuga extract, and an admixture of the Cimicifuga-salt, the coptis and the zanthoxylin accounts for about 1.1% to 80% of the composition by weight and the remaining portion of the composition is the filler material.

### CLAIMING PRIORITY

This is a continuation of the patent application Ser. No. 09/637,162, filed on Aug. 14, 2000.

### BACKGROUND OF THE INVENTION

The present invention relates to an oral care composition applicable to oral hygiene products such as toothpaste and mouth detergent. More particularly, the present invention relates to a composition of salt and herbal extracts for treatment efficacy on periodontal diseases.

Oral care products including dentifrice are known to contain components such as sodium chlorides, antiplasmin agents, allantoin derivatives, vitamines, amino acids and others. Since the selection of the components is substantially influenced by target taste, target flavor and target sweetness, the sodium chloride which is known as effective in oral hygiene has suffered from artificial deformation in composition when used for different products. For example, a peppermint oil and a spearmint oil are used as a flavoring agent to decrease salty taste. Or sodium lauryl sulphate is used as a foaming agent to improve foaming properties. Also, tranexamic acid, aluminium chlorohydroxy allantonate and tocopherol acetate in admixture with sodium chloride are used to treat or prevent periodontal diseases. However, no treatment or prevention effects against periodontal diseases are expected other than slight improvements in taste and flavor. U.S. Pat. No. 5,180,575 discloses a bamboo-salt as a composition for oral care products such as toothpaste. Still, treatment or prevention effects against periodontal disease are hardly expected.

In consideration of the foregoing disadvantages, the prevent inventor has conducted extensive clinical experiments on the combination of salt and herbal extracts for substantial improvement on prevention or treatment effects against periodontal disease while enhancing oral hygiene. Although it took a considerable time period to compare treatment or prevention effects from experimental herbs which are widely being used in oriental herbal treatments, it was eventually discovered that a cimicifuga-salt preferably in admixture of coptis and zanthoxylin produces substantial treatment effects against periodontal diseases such as gingivitis or dental caries.

A cimicifuga-salt consists of a living salt and cimicifuga extracts. The living salt is obtained by melting pure bay salt at a high temperature or preferably at about 1,000° C. for about 48 hours. The living salt is believed to have treatment effect since it reserves condensed energy and osmotic pressure at the melting stage which substantially enhance sterilization effects. Also, it has treatment effects against gum bleeding, edema, inflammation, halitosis, tooth decay and serious periodontal diseases (Donguebogam, Korean Medicinal Book). The nostrum of the living salt is further demonstrated in Sinkum (Living Salt) Therapy for Healthy Life and Living Salt Therapy Sink (by Kyound Jin Park, 1985), and Living Salt Diet for Diabetics, Folklore Living Salt Therapy to Revive Liver Cells (by Il Sun Oh, 1993). The living salt has been used by Oriental doctors specialized in alternative medicine for hundreds of years in Korea.

Commercially available cimicifuga herb contains cimitin C₂₀H₃₄O₇, Et₂O, BuOH, cimicifugin, salicylic acid, cimigenol, 25-O-methylcimigenol-3-xyloside, cimigol, dahurinol, isodahurinol, acerinol, 24-O-acetylacerinol, cimicifugoside, cimicifugenin, 26-O-methylcimifugoside, ciminifugenin A, 26-O-methylcimifugenin A, cimifugenol, friedelin, b-sitosterol, khellol, amminol, 3,4-dimethylcinnamic acid, ferulic acid, iso-ferulic acid, dahurinol, coumarin and others. The cimicifuga herb alleviates pain and inhibits the growth of tuberculosis viruses and dermal fungi in vitro. In the human body, Et₂O serves as sedatives and suppresses edema. BuOH reduces bodily temperature and serves as pain relievers, edema suppressants, and anti-ulceratives.

Consequently, it is understood that the cimicifuga-salt obtained by combining the living salt and cimicifuga extracts is effective for detoxication, fever reduction, anti-inflammation, improvement in the cytoginic function, anti-sepsis, cancer prevention, sterilization, cold symptoms, various anemisa, and hypotension.

Coptis herb is commerically available and includes alkaloid, berberine (4-7%), coptisine, jatoerrhizine, palmitine, magnoflorine, ferulic acid and others. In pharmacological actions, the coptis herb relieves bodily fever, prevents dehydration and toxication. Among the components, the berberine and coptisine are known to serve as antibiotics, laxatives, anti-inflammatory agents and stytics, and stop diarrhea.

Commercially available zanthoxylin is classified to belong to zanthoxylum piperitum and contains sanshool C₁₆H₂₇ON, sanshoon C₁₆H₂₅ON, sanshoamide, geraniol and others. Zanthoxylin serves to warm bodily digestive organs, relieve pain, treat diarrhea and kill intestinal worms. In vitro, it suppresses gram-negatives such as dysentery viruses, and gram-positive aerobic viruses such as staphylococcus aureus and it also kills round worms in swine.

So it is readily understood that the cimicifuga-salt alone or in admixture of coptis and zanthoxylin enhances treatment or prevention effect against periodontal diseases such as gingivitis, dental caries, oral abscess, gum inflammation, tooth decay and other gum or tooth related diseases.

### SUMMARY OF THE INVENTION

The present invention is contrived to overcome the disadvantages in the prior arts. Therefore, it is an object of the present invention to an oral care composition which substantially improves treatment efficacy on periodontal diseases by using a combination of salt and herbal extracts.

To achieve the above-described object, the oral care composition according to the present invention comprises a cimicifuga-salt which consists of a salt and a cimicifuga extract, and wherein the cimicifuga-salt accounts for about 1% to 80% of the composition by weight. The salt and the cimicifuga extract are mixed for the cimicifuga-salt at a weight ratio of about 5:1 to 8:1. The composition containing the cimicifuga-salt may further comprise a sodium chloride, wherein the cimicifuga-salt and the sodium chloride are preferably mixed at a weight ratio of 1:5 to 1:10. The composition may be a toothpaste, a mouth detergent, a mouth wash, a mouth spray, denture cleansing formulation, tooth paints and others. Selectively, the composition may comprise solely the cimicifuga-salt.

In a preferred version, an oral care composition according to the present invention comprises a cimicifuga-salt, a coptis, and a zanthoxylin, wherein the cimicifuga-salt consists of a salt and a cimicifuga extract, and wherein the admixture of the cimicifuga-salt, the coptis and the zanthoxylin accounts for about 1.1% to 80% of the composition by weight. The cimicifuga-salt, the coptis, and the zanthoxylin are mixed for the admixture at a weight ratio of about 50:10:1 to 80:10:1. The composition of the cimicifuga-salt, the coptis, and the zanthoxylin may further comprise a sodium chloride, wherein the admixture of the cimicifuga-salt, the coptis and the zanthoxylin is mixed with the sodium chloride at a weight ratio of 1:4.5 to 1:12. Alternately, the composition may comprise solely the admixture of the cimicifuga-salt, the coptis, and the zanthoxylin.

The advantages of the present invention are: (1) to enable effective herbal extracts to directly apply to interior of a user's mouth together with nostrum living salt in a daily required formulation such as toothpaste, thereby enhancing prevention or treatment effects against periodontal diseases; (2) to make a dentist-allergic periodontal patient give an alternative by allowing him/her to get a convenient access to the healing instead of visiting dental doctors; and (3) to provide a reliable alternative therapy against mouth cancer which is ranked the 8^{th} highest death rate among the U.S. cancer patients.

Although the present invention is briefly summarized, the fuller understanding of the invention can be obtained by the following drawings, detailed description and appended claims.

### THE DETAILED SPECIFICATION OF THE PREFERRED EMBODIMENTS

The oral care composition according to the present invention comprises a cimicifuga-salt. The cimicifuga-salt consists of a salt and a cimicifuga extract, wherein the cimicifuga-salt accounts for about 1% to about 80% of the composition by weight. The salt is a living salt obtained by melting a pure bay salt at about 1000° C. for about 24 hours. The thusly obtained salt is mixed with the cimicifuga extract at a weight ratio of about 5:1 to 8:1, thereby incorporating a cimicifuga-salt. The composition containing the cimicifuga-salt may further comprise a sodium chloride, wherein the cimicifuga-salt and the sodium chloride are preferably mixed at a weight ratio of 1:5 to 1:10. Selectively, the composition may comprise solely the cimicifuga-salt. The composition solely including the cimicifuga-salt may further comprise foaming agents, wetting agents, sweetening agents, flavoring agents, polishing agents, preservatives, binders and pharmacologically active agents.

For a better performance, the oral care composition according to the present invention comprises the cimicifuga-salt, a coptis, a zanthoxylin, and a filler material wherein the admixture of the cimicifuga-salt, the coptis and the zanthoxylin accounts for about 1.1% to 80% of the composition by weight. The cimicifuga-salt, the coptis, and the zanthoxylin are preferably mixed with each other at a weight ratio of about 50:10:1 to 80:10:1. The filler material comprises foaming agents, wetting agents, sweetening agents, flavoring agents, polishing agents, preservatives, binders and pharmacologically active agents. Alternately, the filler material includes a sodium chloride, wherein the admixture of the cimicifuga-salt, the coptis and the zanthoxylin is mixed with the sodium chloride at a weight ratio of 1:4.5 to 1:12. Also, the composition may solely include the cimicifuga-salt, coptis and zanthoxylin. The oral care composition according to the present invention may be incorporated in oral hygiene products such as a toothpaste, a mouth detergent, a tooth powder, a mouth spray, a chewing gum, a gum massage cream, or a denture cleansing formulation. Here, the composition of a cimicifuga-salt alone or in admixture of coptis and zanthoxylin is also called Byczenol (a trademark to be registered by the inventor).

To obtain the best performance, it is recommended that the composition of the cimicifuga-salt alone or in admixture of coptis and zanthoxylin account for about 30% in a classic toothpaste. Selectively, the composition rate can be raised up to 80% for a stronger treatment effect against periodontal diseases. However, the ratio or amount of cimicifuga-salt along or in admixture of coptis and zanthoxylin may be adjusted depending upon treatment or prevention targets.

An effective amount of components for a conventional toothpaste, the filler material, may be mixed with the composition according to the present invention. For example, possible filler materials are polishing agents such as dicalcium, phosphate, silicone dioxide aluminum hydroxide, or calcium carbonate; humectants such as sorbitol, glycerin, or polyethylene glycol; foaming agents such as sodium alkylsulphate, or polyoxyethylene-polyoxypropylene condensation polymer; sweetening agent such as saccharin, or aspartame; flavoring agents such as peppermint oil, or spearmint; preservatives such as methyl paraoxy benzoic acid; therapeutic agents such as sodium fluoride, chlorhexidine, tranexamic acid, or allantoin; binders; and others.

As specified above, The cimicifuga-salt consists of living salt and cimicifuga extracts at a mixing ratio of about 5:1 to 8:1 by weight. The cimicifuga-salt may account for about 1% to 80% of a target composition such as a toothpaste. In a preferred version, the cimicifuga-salt and sodium chloride may be mixed at a weight ratio of about 1:5 to about 1:10 within the target composition.

When the cimicifuga-salt, coptis and zanthoxylin are mixed at a weight ratio of about 50:1:1 to 80:1:1, they preferably account for about 0.1% to 80% by weight within a target composition. Also, when the composition of cimicifuga-salt, coptis and zanthoxylin are mixed with sodium chloride, the preferred weight ratio is about 1:4.5 to 1:12.

The oral care composition according to the present invention will be further described with reference to the accompanying Examples and Comparative Examples.

### COMPARATIVE EXAMPLES 1 TO 4 AND EXAMPLES A, B

Toothpaste components were prepared as shown in Table 1

TABLE 1

| | Comparative Examples | | | | Examples | |
|---|---|---|---|---|---|---|
| Components | 1(%) | 2(%) | 3(%) | 4(%) | A(%) | B(%) |
| Byczenol-A* | — | — | — | — | 10.0 | 20.0 |
| Dicalcium phosphate | 40.0 | 40.0 | 40.0 | 40.0 | 30.0 | 20.0 |
| Non-crystalline sorbitol | 25.0 | 25.0 | 25.0 | 25.0 | 15.0 | 15.0 |
| solution | | | | | | |
| Bamboo-salt | 2.0 | 5.0 | — | — | — | — |
| Sodium chloride | — | — | 1.0 | 1.5 | — | — |
| Aluminum chlorohydroxy | — | — | 0.1 | 0.1 | — | — |
| allantoinate | | | | | | |
| Tocopherol acetate | — | — | 0.1 | 0.1 | — | — |
| Tranexamic acid | — | — | 0.1 | 0.1 | — | — |
| Sodium glutamate | — | — | 0.01 | 0.01 | — | — |
| Sodium alkylsulphate | 2.0 | 2.0 | 2.0 | 2.0 | — | — |
| Sodium saccharin | 1.0 | 1.0 | 1.0 | 1.0 | — | — |
| Sodium carboxymethyl | 1.0 | 1.0 | 1.0 | 1.0 | — | — |
| cellulose | | | | | | |
| Flavoring agent | 0.8 | 0.8 | 0.8 | 0.8 | — | — |
| By adding diluted water, up to | 100 | 100 | 100 | 100 | 100 | 100 |
| U.V. spectrophotometer | 20.0 | 30.0 | 10.0 | 10.0 | 50.0 | 70.0 |
| transmittance | | | | | | |

*Byczenol-A is cimicifuga-salt

### COMPARATIVE EXAMPLES 5 TO 8 AND EXAMPLES C TO F

Toothpaste components were prepared as shown in Table 2

TABLE 2

| | |
|---|---|
| Comparative Examples (%) | Examples (%) |

| Components | 5(%) | 6(%) | 7(%) | 8(%) | C(%) | D(%) | E(%) | F(%) |
|---|---|---|---|---|---|---|---|---|
| Byczenol-B* | — | — | — | — | 25.0 | 40.0 | 55.0 | 70.0 |
| Dicalcium phosphate | 35.0 | 35.0 | 35.0 | 23.0 | — | — | — | — |
| Calcium carbonate | — | — | — | — | — | — | — | — |
| Precipitated silica | — | — | — | — | — | — | — | — |
| Anhydrous silicic acid | — | — | — | — | — | — | — | — |
| Non-crystalline sorbitol solution | — | — | — | — | — | — | — | — |
| Sorbitol solution | — | — | — | — | — | — | — | — |
| Glycerin | — | — | — | — | 35.0 | 30.0 | 20.0 | 15.0 |
| Sodium chloride | 10.0 | — | — | — | — | — | — | — |
| Bamboo-salt | 0.5 | 5.0 | 10.0 | 30.0 | — | — | — | — |
| Tranexamic acid | 0.05 | 0.05 | 0.05 | 0.05 | — | — | — | — |
| Aluminum chlorohydroxy | 0.1 | 0.1 | 0.1 | 0.1 | — | — | — | — |
| allantoinate | | | | | | | | |
| Tocopherol acetate | 0.1 | 0.1 | 0.1 | 0.1 | — | — | — | — |
| 5-amino caproic acid | 0.05 | 0.05 | 0.05 | 0.05 | — | — | — | — |
| Sodium alkysulphate | 2.0 | 2.0 | 2.0 | 2.0 | — | — | — | — |
| Sugar-fatty acid ester | — | — | — | — | — | — | — | — |
| N-acyl glutamate | — | — | — | — | — | — | — | — |
| Magnesium chloride | 0.05 | 0.05 | 0.1 | 0.05 | — | — | — | — |
| Trimagnesium phosphate | 0.05 | 0.05 | 0.05 | 0.05 | — | — | — | — |
| Sodium saccharin | 0.1 | 0.1 | 0.1 | 0.1 | — | — | — | — |
| Methyl Paraben | 0.05 | 0.05 | 0.05 | 0.05 | — | — | — | — |
| Sodium carboxymethyl Cellulose | 0.8 | 0.8 | 0.8 | 0.6 | — | — | — | — |
| Flavoring agent | 0.1 | 0.1 | 0.1 | 0.1 | — | — | — | — |
| By adding diluted water, up to | 100 | 100 | 100 | 100 | 100 | 100 | 100 | 100 |

*Byczenol-B is cimicifuga-salt admixed with coptis and zanthoxylin

### EXPERIMENTAL TEST AND RESULTS THEREOF

Several groups of thirty (30) persons (between age 20 and age 55) suffering from halitosis (1st group) teeth sour (2nd group), gum bleeding (3rd group), gingivitis (4th group) and toothache (5th group) were tested three times a day for fifteen (15) days. The thirty participants brushed their teeth for about three minutes each time during the test period. The first group of thirty persons used the toothpastes containing the composition according to the present invention. The second group of the other thirty persons used conventional tooth pastes as described in the above Comparative Examples. The test results are as shown in Table 3.

TABLE 3

| | | No. of Healed Persons | | | | |
|---|---|---|---|---|---|---|
| Toothpaste | Byczenol | per 30 Participants (%) | | | | |
| Examples | ratio | I* | II* | III* | IV* | V* |
| Example C | 25% | 25 | 26 | 25 | 26 | 26 |
| | | (83.3) | (86.7) | (83.3) | (86.7) | (86.7) |
| Example D | 40% | 26 | 27 | 27 | 27 | 27 |
| | | (86.7) | (90.0) | (90.0) | (90.0) | (90.0) |
| Example E | 55% | 28 | 29 | 28 | 28 | 28 |
| | | (93.3) | (96.7) | (93.3) | (93.3) | (93.3) |
| Example F | 70% | 29 | 30 | 29 | 29 | 29 |
| | | (96.7) | (100.0) | (96.7) | (96.7) | (96.7) |
| Comp. 3 | 0% | 1 | 3 | 2 | 2 | 3 |
| | | (3.3) | (10.0) | (6.7) | (6.7) | (10.0) |
| Comp. 4 | 0% | 0 | 2 | 1 | 3 | 2 |
| | | (0.0) | (6.7) | (3.3) | (10.0) | (6.7) |
| Comp. 5 | 0% | 2 | 0 | 0 | 2 | 1 |
| | | (6.7) | (0.0) | (0.0) | (6.7) | (3.3) |
| Comp. 6 | 0% | 2 | 0 | 1 | 2 | 1 |
| | | (6.7) | (0.0) | (3.3) | (6.7) | (3.3) |

*Type of Participants Healed by Toothpaste Treatment
I: participants with haliosis
II: participants with teeth sour
III: participants with gum bleeding
IV: participants with gingivitis
V: participants with toothache

Table 3 demonstrates treatment effects of the toothpaste containing the oral care composition according to the present invention. As shown therein, the weight of the cimicifuga-salt alone or in admixture of coptis and zanthoxylin (Byczenol) is preferred to account for about 25% to 70% of a toothpaste of conventional components.

Mouth Detergent

| | |
|---|---|
| Ethanol (90%) | 20.0% |
| Glycerine (98%) | 10.0% |
| Polyxyethylene-polyoxypropylene Copolymer | 1.0% |
| Tranexamic acid | 0.05% |
| Byczenol | 10.0% |
| Sodium saccharin | 01% |
| Flavoring agent | 1.0% |
| By adding distilled water up to | 100.0% |

### Example G

### Example H

Mouthwash

| | |
|---|---|
| Sodium bicarbonate | 20.0% |
| Stannic acid | 18.0% |
| Sodium sareosinate-coconut oil | 5.0% |
| Sodium lauryl sulfate | 5.0% |
| Benzalkonium chloride | 2.0% |
| EDTA | 5.0% |
| Sodium tripolyphosphate | 14.0% |
| Polyethylene glycol | 2.0% |
| Byczenol | 24.0% |
| Flavoring agent | 5.0% |

### Example I

Chewing gum

| | |
|---|---|
| Gum base | 15.0% |
| Sorbitol | 30.0% |
| Manniol | 12.0% |
| Glycerine | 13.0% |
| Lecithin | 0.5% |
| Sweetening agent | 2.0% |
| Byczenol | 26.0% |
| Flavoring agent | 1.5% |

### Example J

Gum Massage Cream

| | |
|---|---|
| Glycerol monolaurate | 3.0% |
| Oleic alcohlate | 5.0% |
| Polyethylene glycol | 15.0% |
| White Vaseline | 3.0% |
| Monosodium N-palmitic glutamate | 5.0% |
| Hydroxyethyl cellulose | 5.0% |
| Tocopherol acetate | 0.1% |
| Byczenol | 10.0% |
| Sweetening agent | 0.2% |
| Aluminum chlorohydroxy allantoinate | 3.0% |
| Flavoring agent | 0.3% |
| By adding distilled water up to | 100.0% |

As demonstrated above, the oral care composition according to the present invention prevents or treats periodontal diseases in a reliable healing rate.

An advantage of the present invention is to enable effective herbal extracts to directly apply to interior of a user's mouth together with nostrum living salt in a daily required formulation such as toothpaste, thereby enhancing prevention or treatment effects against periodontal diseases. Another advantage is to make a dentist-allergic periodontal patient give an alternative by allowing him/her to get a convenient access to the healing instead of visiting dental doctors. Further, the oral care composition according to the present invention may be a reliable alternative therapy against mouth cancer which is ranked the 8^{th} highest death rate among the U.S. cancer patients.

Although the invention has been described in considerable detail with reference to certain preferred versions thereof, other versions are possible by converting the aforementioned construction. Therefore, the scope of the invention shall not be limited by the specification specified above and the appended claims.

What is claimed is:

1. An oral care composition comprising a Cimicifuga-salt, a coptis, a zanthoxylin, and a filler material, wherein the Cirmicifuga-salt consists of a salt and a Cimicifuga extract, and wherein an admixture of the Cimicifuga-salt, the coptis and the zanthoxylin accounts for about 1.1% to 80% of the composition by weight and the remaining portion of the composition is the filler material.

2. The composition of claim 1 wherein the Cimicifuga-salt, the coptis, and the zanthoxylin of the composition are admixed with each other at a weight ratio of about 50:10:1 to 80:10:1, wherein the weight of the Cimicifuga-salt is solely variable within the admixture of the Cimicifuga-salt, the coptis, and the zanthoxylin.

3. The composition of claim 1 wherein the filler material comprises a sodium chloride.

4. The composition of claim 3 wherein the admixture of the Cimicifuga-salt, the coptis and the zanthoxylin is mixed with the sodium chloride at a weight ratio of 1:4.5 to 1:12.

5. The composition of claim 4 wherein the composition is a toothpaste.

6. The composition of claim 4 wherein the composition is a mouth detergent.

7. The composition of claim 4 wherein the composition is a mouthwash.

8. The composition of claim 4 wherein the composition is a chewing gum.

9. The composition of claim 4 wherein the composition is a gum massage cream.

10. The composition of claim 1 wherein the composition comprises solely the admixture of the Cimicifuga-salt, the coptis, and the zanthoxylin.

11. The composition of claim 10 wherein the Cimicifuga-salt, the coptis, and the zanthoxylin of the composition are admixed with each other at a weight ratio of about 50:10:1 to 80:10:1, wherein the weight of the Cimicifuga-salt is solely variable within the admixture of the Cimicifuga-salt, the coptis, and the zanthoxylin.

12. The composition of claim 11 wherein the composition is a toothpaste.

13. The composition of claim 11 wherein the composition is a mouth detergent.

14. The composition of claim 11 wherein the composition is a mouthwash.

15. The composition of claim 11 wherein the composition is a chewing gum.

16. The composition of claim 11 wherein the composition is a gum massage cream.
